(19) United States
(12) Patent Application Publication
(10) Pub. No.: US 2005/0137621 A1
(43) Pub. Date: Jun. 23, 2005

(54) PTCA and/or PTA balloon

(75) Inventors: Laurent Stahl, Winterthur (CH); Vitali Verin, Genf (CH); Erwin Berger, Stettfurt (CH); Michael Schwager, Winterthur (CH)

Correspondence Address: BURNS DOANE SWECKER & MATHIS L L P, POST OFFICE BOX 1404, ALEXANDRIA, VA, 22313-1404, US

(73) Assignee: Acrostak Corporation, Winterthur (CH)

(21) Appl. No.: 10/959,428

(22) Filed: Oct. 7, 2004

Related U.S. Application Data
(63) Continuation of application No. PCT/EP03/03304, filed on Mar. 29, 2003.

(30) Foreign Application Priority Data
Apr. 8, 2002 (EP) 02007818.4
Oct. 26, 2002 (EP) 02024013.1

(51) Int. Cl. A61M 29/00

(52) U.S. Cl. 606/194; 604/103.07; 604/916

(57) ABSTRACT

A flexible PTCA and/or PTA device is disclosed which respects the curves of the anatomy for dilatation, centering and setting stents. A method for inflation is also disclosed.

### CROSS REFERENCE TO RELATED APPLICATIONS

This application claims priority under 35 U.S.C. §119 to EP Application 02007818.4 filed in Europe on Apr. 8, 2002 and to EP Application 02024013.1 filed in Europe on Oct. 26, 2002, and as a continuation application under 35 U.S.C. §120 to PCT/EP03/03304 filed as an International Application on Mar. 29, 2003 designating the U.S., the entire contents of which are hereby incorporated by reference in their entireties.

### BACKGROUND

This invention relates to a PTCA and/or PTA device having a balloon for a dilatation catheter, for setting stents and for centering a catheter into the vessel. The invention relates to devices for sutureless anastomosis, too.

Percutaneous coronary angioplasty (“PTCA”) is an established treatment for coronary artery disease. Percutaneous transluminal angioplasty (PTA) is an established treatment for peripheral artery disease. The procedure involves inserting a balloon catheter through a vascular structure-to-structure site at which atherosclerotic plaque has collected on the vessel wall. That means coronary arteries, peripheral arteries and veins, which carry blood. The plaque is disrupted by inflating the balloon at the distal end of the catheter, thereby increasing the intraluminal diameter of the vascular structure and spreading or loosening the plaque. Disruption of the plaque ultimately reduces the restriction to the blood flow through the vascular structure. After sufficient expansion, the balloon is then deflated and removed, and the area of disruption heals.

While the PTCA and/or PTA technique is very widely used, one problem, which limits its acceptability, is a possible consequence known as restenosis. In hopes of preventing abrupt closures and restenosis, the stents techniques were developed. Such stents are tubular devices, which provide structural support for maintaining a vessel open.

Procedures used for stent deployment in a vessel generally involve the introduction of a stent, in a crimped condition onto a balloon catheter into a vessel and the optimal localizations of the stent relative to the intended implantation or target site, followed by the expansion of the stent through inflation of the balloon such that is locked in the desired position in apposition to the vessel wall. Certain stents require an ancillary means for expansion. For example, a stent may be fitted over a deflated angioplasty balloon, which is then introduced into the vessel and inflated, thereby expanding the stent and deploying it correctly. The procedure of setting stents incorporates the following steps: An adequate channel for passage of the balloon stent assembly is created by inflating a first balloon without a stent within the stenosed region. Then the balloon stent assembly is advanced into the target vessel, the crimped stent is localized and optimally positioned relative to the intended implantation site in the stenosis, and the stent is expanded by inflating the “carrier” balloon, so as to achieve contact between the stent and the walls of the vessel. After that step the axially symmetric tubular geometry of the stent and uniform circumferential contact of the stent with the walls of the vessel were optimized by inflating another balloon capable of withstanding relatively high distending pressures within the deployed stent. In order to avoid damage to the target vessel adjacent to an implanted stent, the balloon used for post-dilatation is optimally of the same length or shorter than the stent.

The patent U.S. Pat. No. 5,213,361 describes a device for preventing restenosis after angioplasty comprising, among various embodiments, a catheter having a balloon at its distal end and a centre core or tube in which a conventional guide wire is receivable. Particles or crystals of radioactive material are embedded in or mounted on the tube inside the balloon and a retractable shielding sleeve is slidable along the tube to cover the radioactive source, blocking exposure to radiation until it is shifted away. Such a structure is said to allow radiation of a vascular structure immediately following completion of angioplasty, without separately inserting a radiation source.

Standard dilatation balloons are not well-suited transport and to take up radioactive radiating sources because the centre core or guide wire lumen tends to warp on the stretch inside the balloon, thereby forming an undulated line. The radioactive radiation source, however, has to be centered as exactly as possible inside the vessel in order to avoid the vessel wall being burned.

The document U.S. Pat. No. 5,976,106 describes a balloon catheter comprising a catheter tube surrounded distally by an elongated inflatable balloon. Throughout the catheter tube is a longitudinal lumen for positioning a radioactive radiation emitter within the balloon. The catheter comprises a second lumen for directing inflation fluid into the balloon. Waist means creating a waist are mounted on the balloon to squeeze it down to nearly the diameter of the catheter thereby leaving a small passage for the inflation fluid. The waist means divide the balloon into sections to assure a close centre fit of the catheter within the balloon.

The document U.S. Pat. No. 5,910,101 discloses an intravascular catheter having an expandable inflation region adapted for centering a radiation dose in a body lumen for a period of time sufficient to permit delivery of a radiation dose to the body lumen. The catheter includes a delivery lumen, and a blind internal lumen for receiving a wire having a radiation source located at the distal end of the wire. The blind internal lumen is received in the delivery lumen of the catheter. The blind internal lumen prevents contamination of the radiation source by fluids from the body lumen. The radiation source is advanced through the blind internal lumen towards the inflation region of the catheter. The inflation region includes a plurality of balloon lobe at the distal end of the catheter. The plurality of balloon lobe, when inflated, can centre the radiation source within a curved section of the body lumen.

### SUMMARY

A PTCA and/or PTA device having a balloon is disclosed with a sufficient flexibility for use in sinuous coronary arteries for PTCA balloon or sinuous peripheral arteries for a PTA balloon. The balloon is, after inflation, flexible enough and follows during the placement the largest and/or smallest curvatures. Very short balloons with a waist can be used for preventing a slippery effect (which is the typical reaction of a known short balloon) during the inflation phase of the balloon. The shorter the balloon the smaller is the section of the vessel inner wall damage. Ideal positioning can be achieved in a stenosis of the coronary and/or peripheral arteries.

The known meaning of the term “compliance” is the functional relation between the pressure inside the balloon and the outer diameter of the balloon.

The meaning in the invention of the term “compliance” is the relation between the momental pressure in the balloon and its momental diameter during inflation.

Depending on the use of the inventive balloon, the compliance can be adjusted. This characteristic reduces the damaging of the vessel innerwall and gives an anti-slippery effect to the balloon. A more precise location by the stenosis is attainable.

The PTCA and/or PTA inflation balloon has at least one balloon lobe. When the balloon has more than one lobe then at least one waist is situated between the lobes. The lobes and the waists are made from plastic. No means (for example rings etc) are necessary for making the waists. After the inflation process the ends of the balloon near the waists don't have any fold, which is a disadvantage in the state of the art. Preferably the balloon has 2 till 5 lobes in line. The usual total length of the PTCA and/or PTA inflation balloon is in the range of 6 mm to 50 mm, preferably from 8 to 40. The length of a balloon lobe is in the range of 3 mm to 40 mm, preferably from 4 to10 mm. An inline of balloons, which total length is either longer or shorter than balloons known in the prior art, can be used. In the state of inflation the balloon remains flexible along a vessel curvature or sinuosity.

The PTCA inflation balloon can have a diameter of the lobe in the range from 1 mm to 5 mm, preferably from 2 mm to 4 mm. The diameter of the waist can be in the range from 1 mm to 2 mm, preferably from 1.1 mm to 1.5 mm. The PTCA inflation balloon can work at a pressure range from 4 atm to 30 atm, preferably from 5 atm to 15 atm.

The PTA inflation balloon can have a diameter of the lobe in the range from 1 mm to 30 mm, preferably from 2 to 15 mm. The diameter of the waist can be in the range from 1 mm to 5 mm, preferably from 1.1 to 4.5 mm. The PTA inflation balloon can work at a pressure range from 4 atm to 30 atm, preferably from 5 atm to 25 atm.

The diameters of the single lobe inside the balloon can be different. For some exemplary PTCA and/or PTA balloons it is necessary that the balloon lobes have different sizes. The distal balloon lobe is smaller and/or larger than the proximal balloon lobe.

To achieve a right positioning of the balloon at the plaque location, the balloon can have at least two knobs. The knobs are arranged near both ends of the balloon. Knobs can be arranged along the circumference of a balloon.

The knobs on the lobes and/or the waists solve two problems. On the one hand the knobs prevent a shifting in axial direction. On the other hand the vessel connector which is situated as known over the balloon is inflated regularly and without creases. That means the knobs provide a positive effect in radial direction, too.

The method for inflating the PTCA and/or PTA inflation balloon has at least two phases. For example, in the first phase of the balloon inflation the waist does not inflate and dilate and has an anti-slippery effect (because the stenosis will be blocked into the waist of the balloon), in the second phase the waist will disappear during the further balloon inflation preferably, for example, at pressure from 8 atm to 12 atm.

The non slippery effect due to knobs placed onto the balloon is ideal when using a stent balloon assembly. The stent crimped onto the balloon is better fixed on the balloon through the knobs.

The PTCA and/or PTA inflation balloon can be appropriate for dilatation, bifurcation dilatation, centering, stenting, osteal stenting, bifurcation stenting applications.

Preferably the balloon has, for example, an outer tube and an inner tube whereas the outer tube is welded with the proximal end of the balloon, whereas the inner tube is welded with the distal end of the balloon and whereas the outer tube is welded at least one position with the inner tube in that way that hollow spaces are generated along the axis for inflation and/or deflation the balloon. An advantage of that arrangement is preventing a shifting between the inner tube and the outer tube.

Furthermore it is possible that the welding point of the inner tube with the distal end of the balloon is in the inner range of the balloon. An advantage is preventing damages at the inner wall of the vessel by the end of the inner tube of the balloon.

### BRIEF DESCRIPTION OF THE DRAWINGS

Additional objects and features of the invention will appear from the following description in which the preferred embodiments are set forth in detail in conjunction with the accompanying drawings, wherein:

FIG. 1 is a longitudinal cut of an exemplary PTCA dilatation balloon;

FIG. 2 is an exemplary balloon with five lobes;

FIG. 3 is an exemplary diagram for explaining the term “compliance”;

FIG. 4 is an exemplary two lobe balloon, situated near a plaque position;

FIG. 5 is an exemplary two lobe balloon with different lobe diameters;

FIG. 6 is an exemplary balloon with knobs FIG. 6a-6c are different shaped knobs;

FIG. 7 is an exemplary lobe of a stent assembly;

FIG. 8 is an exemplary balloon with an inner and outer tube;

FIG. 9 is an exemplary two lobe balloon with partially welded inner and outer tube;

FIG. 10 is a cross section A-A of FIG. 9; and

FIG. 11 is an exemplary balloon wherein the distal welding point is situated in the inner range of the lobe.

### DETAILED DESCRIPTION

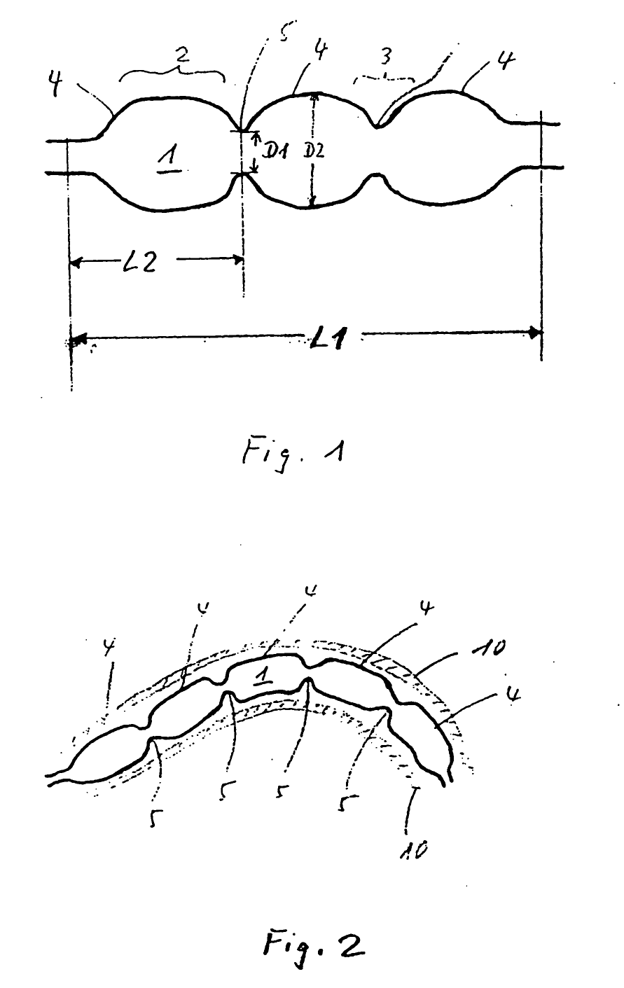

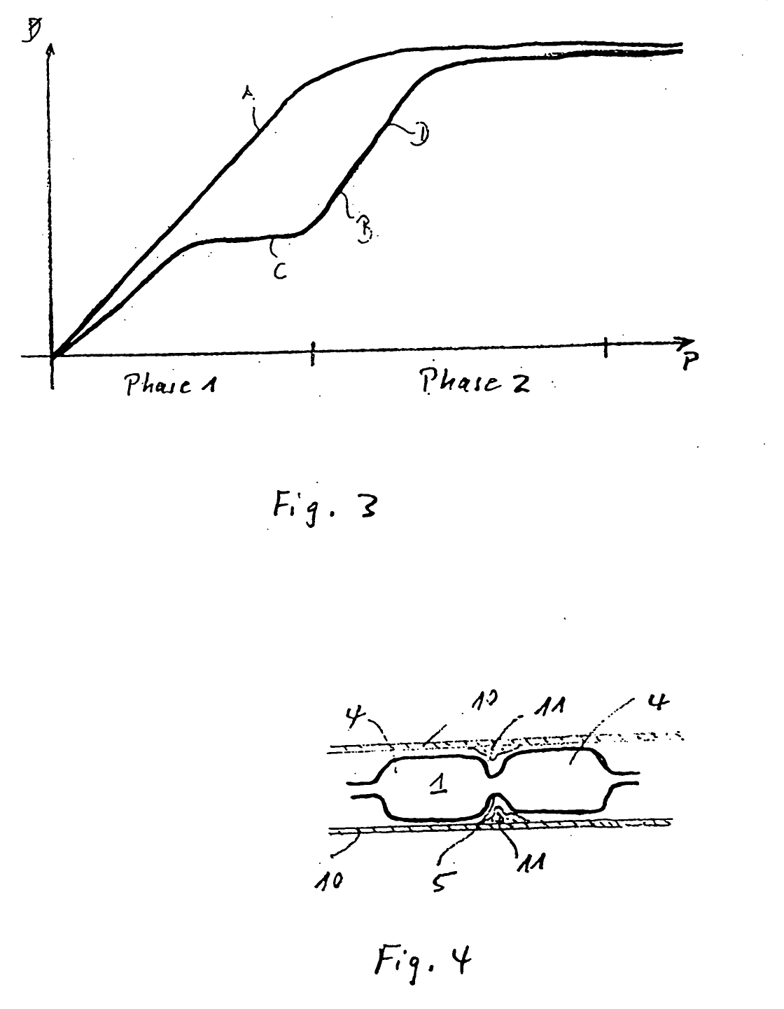

Referring to FIG. 1, the balloon 1 has three balloon lobes 4 with two waists 5. The regions 2, the lobe, and the regions 3, the waist, have a different compliance shown in FIG. 3. D1 is the outer diameter of the waist and D2 is the outer diameter of the whole balloon 1 or the balloon lobe 4. The length L1 is the total length of the whole balloon 1 and L2 is the length of the balloon lobe 4.

FIG. 2 shows an assembly of a PTCA balloon 1 with five lobes 4 and four waists 5 in line along a vessel 10 like arteries. The balloon is made for curved and sinuous vessels 10.

The diagram in FIG. 3 explains the term “compliance”, as the relation between the momenta pressure in the balloon and its momenta diameter during inflation. The curve A shows a normal behavior of a balloon known from the prior art. The diagram B shows a typical two phase method for inflating the PTCA and/or PTA inflation balloon. In the plateau C of phase 1 the waist does not inflate and dilate and has an anti-slippery effect, in phase 2 the waist will disappear during the further balloon inflation D preferably at pressure from 8 atm to 12 atm.

This behavior can reduce damage of the vessel inner walls. Furthermore, the invention makes possible a more precise location of the waist exactly at the plaque/stenosis of the vessel 10.

FIG. 4 shows a PTCA and/or a PTA balloon 1 with two lobes 4 which are situated near a position at the plaque 11 in a vessel 10. The central waist 5 will first have an anti-slippery effect at low pressure (for example under about 8 atm till 10 atm) and will disappear while inflating the balloon 1 with more pressure, as shown in FIG. 3.

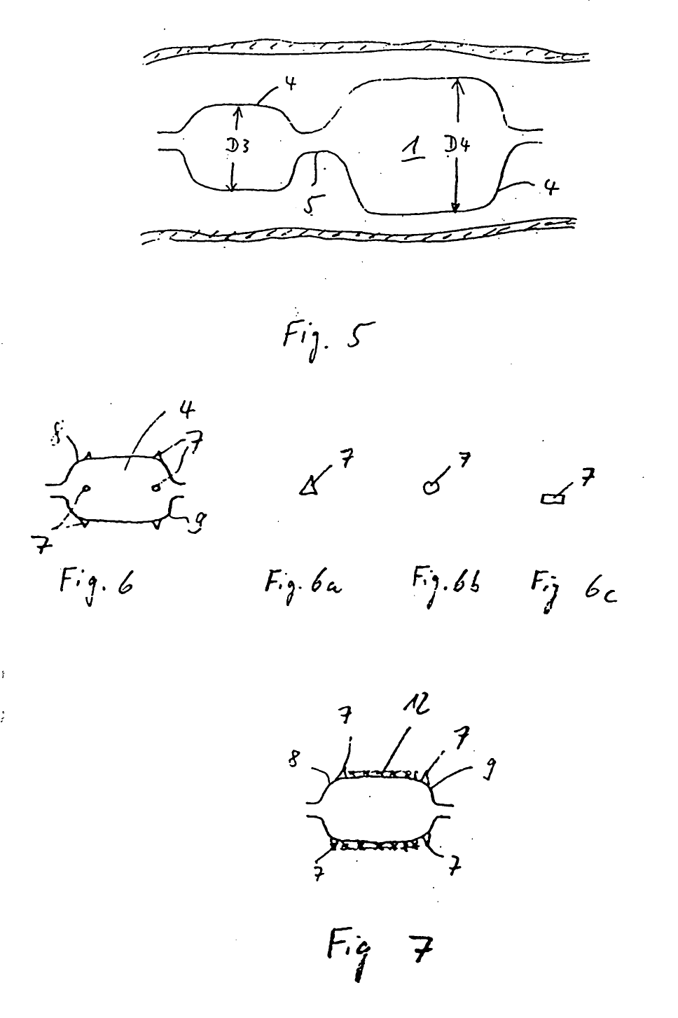

A special application of a PTCA balloon 1 is shown in FIG. 5. The balloon 1 has two lobes 4 with different diameters D3 and D4. This type of balloon 1 is ideal for bifurcation dilatation. Knobs 7 (FIG. 6) can be used to prevent the balloon from slipping out of the stenosis. One lobe 4 can be arranged with knobs 7. Knobs 7 can be differently shaped as shown in the FIGS. 6a, 6b and 6c. The knobs 7 can be situated at many different places on the surface of the balloon lobe 4, e. g. two knobs 7 are arranged at the ends 8 and 9 and/or knobs are arranged around the circumference of the lobe 4. Other embodiments are possible. The embodiment referred to as knobs 7 is shown in the FIGS. 6,6a, 6b and 6c. The knobs 7 can be situated at any desired location on the waists 5, including locating proximally and/or distally.

FIG. 7 shows a lobe of a stent balloon assembly. The stent 12 is arranged between the knobs 7. The configuration of knobs 7 secures the stent during the movement of the stent balloon assembly within the vessel.

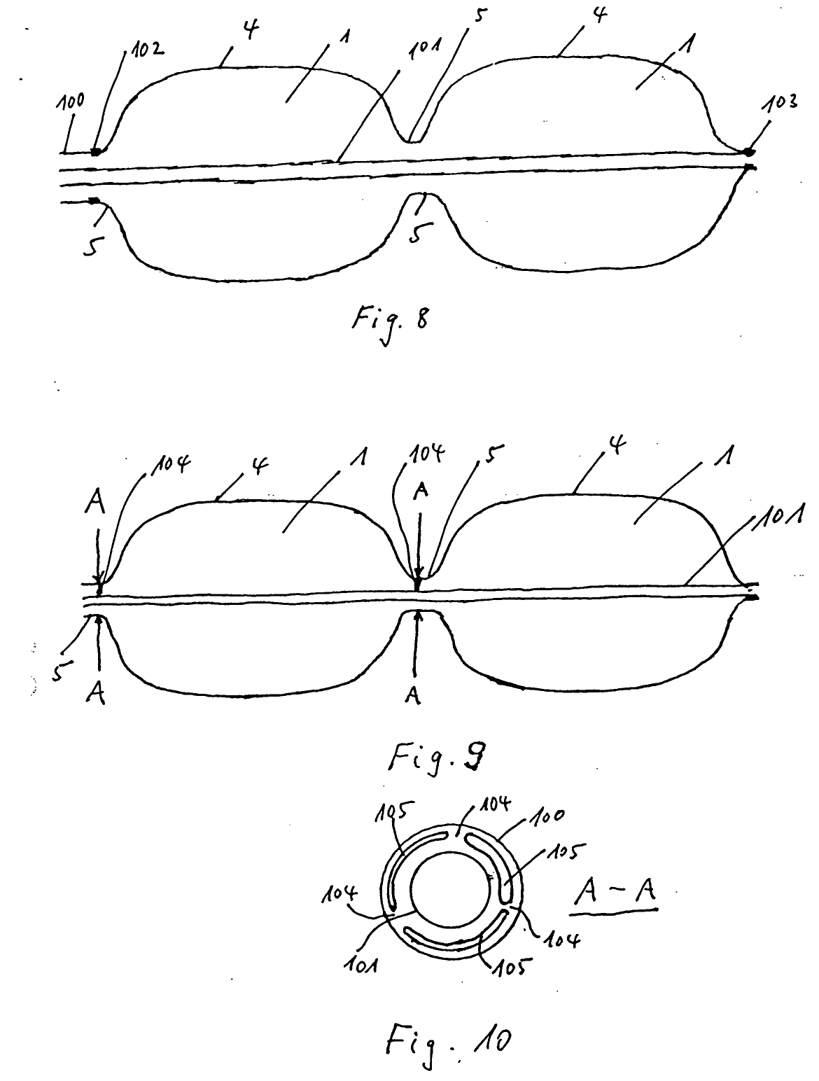

FIG. 8 shows a PTCA and/or PTA inflation balloon 1 with two lobes 4. An inner tube 101 is welded with the distal end 103 of the last lobe 4. An outer tube 100 is welded with the proximal end 102 of the first lobe 4.

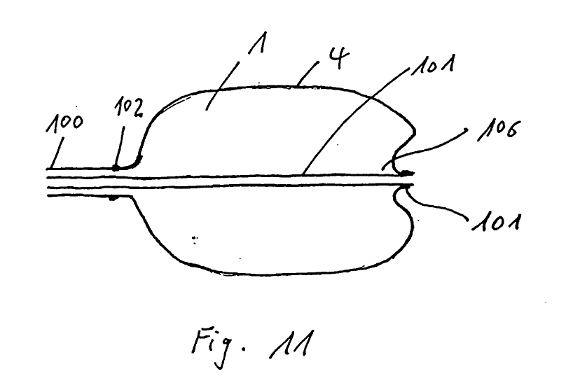

FIG. 9 shows generally the same assembly as in FIG. 8. The difference is the welding of the inner tube 101 with the waists 5 and/or the outer tube 100. The outer tube 100 is welded with the proximal end 102 of the balloon 1 and the waist 5. The inner tube 101 is welded with the distal end 103 of the balloon 1. It is also possible that the outer tube 100 is welded at least at one position 104 with the inner tube 101. In an exemplary embodiment, hollow spaces 105 (FIG. 10) are generated along the axis for inflation and/or deflation of the balloon 1. These hollow spaces 105 with the welding positions 104 are shown in FIG. 10, along cross section A-A of FIG. 9.

FIG. 11 shows a PTCA and/or PTA inflation balloon 1 with one lobe 4, wherein the welding point of the inner tube 101 with the distal end 103 of the lobe 4 is in the inner range 106 of the lobe 4.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. PTCA and/or PTA inflation device for a catheter comprising:
a balloon; and
at least one region of the balloon with a defined adjustable compliance.

2. PTCA and/or PTA inflation device for a catheter, comprising:
a balloon; and
at least two regions with at least two different defined compliances.

3. PTCA and/or PTA inflation device according to claim 2, comprising:
at least two balloon lobes with a waist between the balloon lobes.

4. PTCA inflation device according to claim 3 having a diameter (D1) of the waist in a range from 1 mm to 2 mm.

5. PTA inflation device according to claim 3 having a diameter (D1) of the waist in a range from 1 mm to 5 mm.

6. PTCA inflation device according to claim 4 having a diameter (D2) of the balloon lobes in a range from 1 mm to 5 mm.

7. PTA inflation device according to claim 5 having a diameter (D2) of the balloon lobes in a range from 1 mm to 30 mm.

8. PTCA and/or PTA inflation device according to claim 3, wherein diameters (D3, D4) of the balloon lobes are different.

9. PTCA and/or PTA inflation device according to claim 1, having a total length (L1) in a range 6 mm to 50 mm.

10. PTCA and/or PTA inflation device according to claim 3, wherein the balloon lobes have a length (L2) in a range of 3 mm to 40 mm.

11. PTCA and/or PTA inflation device according to claim 1, having no more than 5 balloon lobes in line.

12. PTCA and/or PTA inflation device according to claim 1, having at least two knobs.

13. Method for inflating the PTCA and/or PTA inflation device of claim 1, comprising:
a first phase for inflating the balloon; and.
a second phase for inflating the balloon.

14. Method for inflating the PTCA and/or PTA inflation device according to claim 13, comprising:
applying the inflation device for at least one of dilatation, bifurcation dilatation, centering, stenting, osteal stenting, and bifurcation stenting.

15. PTCA and/or PTA inflation device according to claim 1, comprising:
an outer tube; and
an inner tube wherein the outer tube is welded with a proximal end of the device, the inner tube is welded with a distal end of the device, and the outer tube is welded in at least one position with the inner tube such that hollow spaces are generated along an axis for inflation and/or deflation of the device.

16. PTCA and/or PTA inflation device according to claim 15, wherein the welding point of the inner tube with the distal end of the device is in an inner range of the device.

17. PTCA inflation device according to claim 3 having a diameter (D1) of the waist in a range from 1.1 mm to 1.5 mm.

18. PTA inflation device according to claim 3 having a diameter (D1) of the waist in a range from 1.1 mm to 4.5 mm.

19. PTCA inflation device according to claim 4 having a diameter (D2) of the balloon lobes in a range from 2 mm to 4 mm.

20. PTA inflation device according to claim 5 having a diameter (D2) of the balloon lobes in a range from 2 mm to 15 mm.

21. PTCA and/or PTA inflation device according to claim 1, having a total length (L1) in a range 8 mm to 40 mm.

22. PTCA and/or PTA inflation device according to claim 3, wherein the balloon lobes have a length (L2) in a range of 4 mm to 10 mm.
